(19) United States
(12) Patent Application Publication
(10) Pub. No.: US 2023/0123659 A1
(43) Pub. Date: Apr. 20, 2023

(54) MAPPING CRITICAL BRAIN SITES USING INTRACRANIAL ELECTROPHYSIOLOGY AND MACHINE LEARNING

(71) Applicants: Northwestern University, Evanston, IL (US); The Trustees of Indiana University, Bloomington, IN (US)

(72) Inventors: Marc W. Slutzky, Northbrook, IL (US); Jason Kenneth Hsieh, Cleveland, OH (US); Richard F. Betzel, Bloomington, IN (US); Matthew Christopher Tate, Wilmette, IL (US)

(21) Appl. No.: 17/966,286

(22) Filed: Oct. 14, 2022

Related U.S. Application Data
(60) Provisional application No. 63/255,677, filed on Oct. 14, 2021.

(51) Int. Cl. A61B 5/00; A61B 5/377; A61B 5/374

(52) U.S. Cl. CPC A61B 5/0042; A61B 5/377; A61B 5/374

(57) ABSTRACT

A system for performing functional brain mapping includes a memory configured to store first data from a magnetic resonance imaging (MRI) system and second data from electrodes. The system also includes a processor operatively coupled to the memory and configured to identify first edges in a brain network based on the first data from the MRI and second edges in the brain network based on the second data from the electrodes. The processor is configured to determine, based on the first edges and the second edges, connectivity metrics for the brain network. The processor is also configured to generate, based at least in part on the connectivity metrics, a decoder that differentiates between critical nodes and non-critical nodes in the brain network.

### CROSS-REFERENCE TO RELATED APPLICATION

The present application claims the priority benefit of U.S. Provisional Patent App. No. 63/255,677 filed on Oct. 14, 2021, the entire disclosure of which is incorporated by reference herein.

### BACKGROUND

Functional brain mapping (FBM) refers to techniques used to identify and map portions of an individual's brain that are critical to “eloquent” functions such as speech, language, sensorimotor function, etc. Functional brain mapping is generally performed on people with epilepsy, brain tumors, etc. who require removal of the affected areas of the brain. By using FBM to determine the areas of the patient's brain which are critical to these eloquent functions (i.e., critical nodes in the brain network), the surgeon is able to perform the surgery in a manner that is most likely to prevent postoperative functional loss. Traditional functional brain mapping is an invasive process that uses direct electrical stimulation to determine brain areas that are involved in these critical functions. The electrical stimulation can cause adverse effects including seizures, which can cause significant discomfort or morbidity for the patient, and also can miss some critical areas.

### SUMMARY

An illustrative system for performing functional brain mapping includes a memory configured to store first data from a magnetic resonance imaging (MRI) system and second data from one or more electrodes. The system also includes a processor operatively coupled to the memory and configured to identify first edges in a brain network based on the first data from the MRI and second edges in the brain network based on the second data from the one or more electrodes. The processor is configured to determine, based on the first edges and the second edges, network connectivity metrics for the brain network. The processor is also configured to generate, based at least in part on the network connectivity metrics, a decoder that differentiates between critical nodes and non-critical nodes in the brain network.

In some embodiments, the MRI data includes diffusion MRI data and the one or more electrodes comprise stereo-electroencephalography electrodes. In other embodiments, the MRI data includes BOLD signal (functional MRI) and the one or more electrodes comprise electrocorticography electrodes. In one embodiment, the processor is configured to generate a brain function map with the decoder, where the brain function map depicts the critical nodes and the non-critical nodes. In another embodiment, the processor is configured to predict locations of critical nodes for a new patient based on the decoder. In other embodiments, the processor performs tractography on the first data to identify the first edges in the brain network. In one embodiment, the processor performs voxel parcellation and electrode co-registration on the first data.

The processor can also be configured to determine first network connectivity metrics based on the first data and second network connectivity metrics based on the second data. In one embodiment, the processor is configured to determine third network connectivity metrics based on third data from the MRI system and fourth network connectivity metrics based on fourth data from the one or more electrodes, where the first data is diffusion MRI data, the second data is stereo-electroencephalography electrode data, the third data is functional MRI data, and the fourth data is electrocorticography electrode data. In another embodiment, the processor combines the first network connectivity metrics, the second connectivity network metrics, the third network connectivity metrics, and/or the fourth network connectivity metrics. In one embodiment, the network connectivity metrics are static, dynamic, or based on time-averaged data. In another embodiment, the network connectivity metrics include one or more of local efficiency, participation coefficient, and clustering coefficient.

An illustrative method of performing functional brain mapping includes storing, in a memory, first data from a magnetic resonance imaging (MRI) system and second data from one or more electrodes. The method also includes identifying, by a processor in communication with the memory, first edges in a brain network based on the first data from the MRI and second edges in the brain network based on the second data from the one or more electrodes. The method also includes determining, by the processor and based on the first edges and the second edges, network connectivity metrics for the brain network. The method further includes generating, by the processor and based at least in part on the network connectivity metrics, a decoder that differentiates between critical nodes and non-critical nodes in the brain network.

In one embodiment, the method also includes generating, by the processor, a brain function map with the decoder. In an illustrative embodiment, the brain function map includes a plurality of nodes, and the method further includes identifying, by the processor, one or more of the nodes as critical nodes. The method can also include identifying, by the processor, the critical nodes as language error (LE) nodes or speech arrest (SA) nodes. The method can also include identifying, by the processor, nodes critical to motor or sensory function. In another embodiment, the method includes calculating correlations between high-gamma activity on each electrode (node) to identify the edges between the nodes. In some embodiments, the method also includes recording, by the processor, local field potentials during a task performed by a patient, where the high-gamma correlations are calculated based at least in part on the local field potentials.

The method can also include predicting, by the processor, locations of critical nodes for a new patient using the decoder. In one embodiment, identifying the first edge in the brain network comprises performing, by the processor, tractography on the first data to identify the first edges. The method can further include performing, by the processor, voxel parcellation and electrode co-registration on the first data.

Other principal features and advantages of the invention will become apparent to those skilled in the art upon review of the following drawings, the detailed description, and the appended claims.

### BRIEF DESCRIPTION OF THE DRAWINGS

Illustrative embodiments of the invention will hereafter be described with reference to the accompanying drawings, wherein like numerals denote like elements.

FIG. 1A depicts how DES is used either intraoperatively (depicted) or in the epilepsy monitoring unit to identify sites critical to language and speech in accordance with an illustrative embodiment.

FIG. 1B depicts ECoG recorded continuously during a simple word-reading task performed by each subject in accordance with an illustrative embodiment.

FIG. 1C depicts how a single, static network for each patient was generated using the correlations between high gamma activity on each electrode in accordance with an illustrative embodiment.

FIG. 2A is a histogram of the number of communities per patient in accordance with an illustrative embodiment.

FIG. 2B depicts coassignment percentages and chance coassignment in accordance with an illustrative embodiment.

FIG. 2C shows network metrics for critical (LE+SA) nodes vs. other nodes in accordance with an illustrative embodiment.

FIG. 2D depicts network metrics for LE nodes and SA nodes vs. other nodes in accordance with an illustrative embodiment

FIG. 2E is a first diagram explaining coassignment and other network metrics in accordance with an illustrative embodiment.

FIG. 2F is a second diagram explaining coassignment and other network metrics in accordance with an illustrative embodiment.

FIG. 3A depicts all electrodes from all patients on a single template brain in accordance with an illustrative embodiment.

FIG. 3B depicts three example patient brain networks in accordance with an illustrative embodiment.

FIG. 3C depicts network communities (in different shades of gray) from the three example patients in accordance with an illustrative embodiment.

FIG. 3D depicts network metrics for the three example patients in accordance with an illustrative embodiment.

FIG. 4 depicts balanced accuracy, sensitivity, and ROC curves for within-subject (top row) and across-subject (bottom row) classification in accordance with an illustrative embodiment.

FIG. 5 is a flow diagram depicting operations performed by the system in accordance with an illustrative embodiment.

FIG. 6 is a flow diagram depicting operations performed by the system in accordance with an illustrative embodiment.

FIG. 7 depicts a computing device for performing functional brain mapping in accordance with an illustrative embodiment.

### DETAILED DESCRIPTION

Individuals with brain tumors, epilepsy, vascular malformations or aneurysms, or other lesions near critical areas of the brain, etc. often require mapping of areas of the cerebral cortex that are critical to control of important functions, such as movement, sensation, speech and language. This is necessary for the neurosurgeon to optimally resect the maximum amount of brain tissue while preserving as much brain function as possible. These functional areas of the brain can be considered critical nodes in a larger brain network related to motor and language functions.

Historically, important abilities such as speech and language have been viewed as localized to focal areas of the cortex. Moreover, direct electrocortical stimulation (DES) has long been used to identify focal sites thought to be critical to eloquent brain functions. Yet, more recent studies have shown that large cortical networks are activated during language and motor tasks, leading many to hypothesize that eloquent functions may instead be emergent properties of distributed brain networks. The inventors sought to reconcile these different viewpoints and elucidate the network properties of critical cortical sites, or nodes.

Direct electrocortical stimulation (DES) has been used in functional brain mapping for over 80 years. However, DES has risks, and can sometimes miss areas of the brain that are critical to function. In addition, DES performed intraoperatively increases risk to the patient due to prolonged surgical time. Described below are methods and systems that may reduce the need for DES, which could reduce surgical time. For example, the proposed methods and systems may augment or replace DES to provide better prediction of surgical outcomes than DES alone, which enables further improvement of functional outcomes. In addition, the proposed techniques may allow physicians to move beyond motor, sensory, and language mapping, and open the field to better map more complex cognitive functions, such as memory and spatial attention, which are not currently mapped for surgery because they are too difficult to directly interrogate with stimulation.

Electrocorticography (ECoG) was used to investigate the network signatures of nodes predicted to be critical to language function by DES. A single, static language network was generated for each of sixteen patients who underwent either awake craniotomy for brain tumor resection, or extraoperative monitoring using implanted subdural electrode grids for epileptogenic focus localization, using high-gamma correlations calculated from local field potentials recorded during a simple word-reading task. Modularity maximization was used to find the community membership of network nodes, and several network metrics were calculated.

As part of the research, the inventors discovered different network signatures for nodes that caused speech arrest and language errors when stimulated. Both types of critical node were characterized by a lower clustering coefficient, local efficiency, and eigenvector centrality than other nodes. However, nodes responsible for language errors, in particular, exhibited higher participation coefficients. Taken together, these metrics form a network signature that strongly implicates language-critical nodes as connectors in the language network. The inventors were able to use this limited set of network features alone to train simple machine learning classifiers to predict which nodes would be critical to speech and language. When training these classifiers on each individual patient, balanced accuracy approached 80% with a sensitivity near 90%. These classifiers were further applied across patients, using leave-one-patient out cross-validation, and balanced accuracies above 70% were achieved with a sensitivity over 80%.

These findings suggest that a node's pattern of connections within the language network substantially helps to inform its importance to function. For higher-order cognitive functions such as language, which depend on coordinated actions of multiple subnetworks (communities), the connectors between these nodes are critical to function. In contrast, for lower-order functions, such as speech articulation (identified via speech arrests), or possibly for motor function, connectors between communities are less critical.

Intraoperative functional cortical mapping using direct electrocortical stimulation (DES) was described by Penfield in the 1930s and later adapted to language by Ojemann in the 1970s. DES is used to identify cortical sites that are critical to a given function—mainly motor movement, sensation, speech, and language. DES enables surgeons to avoid resecting these sites and thereby reduces functional deficits after surgical resections of epileptogenic foci, tumors, and vascular malformations. As such, DES has remained the gold standard for functional brain mapping for nearly a century, although the exact effects of DES on brain activity are not clearly understood. Studies based on stimulation, along with those based on lesions have supported the paradigm that many eloquent cortical functions, such as language, may be sequestered in highly localized brain areas.

More recent studies support the activation of broad cortical networks during directed brain activity. During language, both temporal and extra-temporal multi-areal networks may participate in different perceptual, preparatory, and productive subtasks. It is not clear what is special about the sites identified as critical to language or speech by DES when broad networks are active during the perception, processing, planning, and production of speech and language.

Graph theoretic approaches are a powerful and flexible means of studying brain networks. When the connections (edges) in these networks are estimated using measurements of neural activity (e.g., blood-oxygen dependent signals or electric fields), the resultant network models are said to reflect functional connectivity in the brain. In contrast, structural connectivity relies upon the study of physical connections (or their imaging correlates) between neurons or brain regions. Magnetic resonance imaging (MRI) studies have suggested the presence of multiple modules (a.k.a. communities) in functional brain networks that reorganize dynamically according to task demands. This reorganization is dependent on specialized and flexible connector that adaptively reconfigure between a variety of task states and help integrate information between relatively more stable brain network modules. Lesions to these connectors from, e.g., strokes, led to greater changes in network organization than damage to nodes important for within-module communication (local hubs).

Experimental inhibition of these connector nodes—and not neighboring nodes—can cause disruption during working memory tasks. On the other hand, simulated lesions of more globally connected nodes also have been shown to have widespread effects on the network. It is not clear how these network properties might be predictive of DES effects and on language function in particular.

To develop the proposed methods and systems, the inventors examined the network properties of functional language networks using electrocorticography (ECoG). In one embodiment, high-gamma (Hγ, 70-200 Hz) activity from ECoG recorded during a language task was used to investigate the network signatures of nodes predicted by DES to be critical to speech and language. Specifically, measures of node-based global and local connectivity within language networks was assessed. Further, it was demonstrated that these critical nodes could be predicted by their network signatures alone.

The methods and systems described herein were developed in part based on a study that was conducted in patients with medically intractable epilepsy who required invasive ECoG monitoring for clinical treatment of their epilepsy and patients who required awake craniotomy and functional mapping for the resection of brain tumors. Included patients were found by DES to have cortical sites critical to speech or language. Subjects with tumor-related symptoms affecting speech production (as determined by neuropsychological assessment) and nonnative English speakers were excluded from the study.

As per the standard of care for awake craniotomies, patients with brain tumors were first anesthetized with low doses of propofol and remifentanil, then awakened for direct cortical stimulation mapping. All experiments were performed after cortical stimulation, hence, during experiments, no general anesthesia had been administered for at least 45 minutes. As a result, no effects on speech or language were detected. After functional mapping, 8×8 electrode grids with 4-millimeter (mm) interelectrode spacing were placed over the posteroventral frontal cortices (precentral and inferior frontal gyri), and occasionally over the superior temporal/angular gyri as well. All tumors were located at least two gyri (2-3 centimeters (cm)) away from the recording electrodes. In participants with epilepsy, standard clinical ECoG grids and strips were placed according to clinical necessity. All patients in this study were left hemisphere-dominant and had left-sided temporal, frontal, or parietal subdural electrode coverage.

Functional mapping was performed using DES according to standard of care by the neurosurgery (for intraoperative mapping, using an Ojemann stimulator) or epilepsy (for extraoperative mapping in the epilepsy monitoring unit) teams. Although the specific tasks used for functional language mapping varied by context (intra- vs. extra-operative mapping), in general, patients were asked to name, repeat, speak spontaneously, or perform a comprehension task while high-frequency stimulation was intermittently applied to different cortical regions. In this study, DES functional mapping results were considered the ground truth. Regions of cortex that mapped positive for language or speech function were defined to be critical nodes, and these were further subdivided into language error (LE) nodes and speech arrest (SA) nodes. Areas where stimulation resulted in reproducible cessation of speech during every task modality were defined as SA nodes, and all other critical nodes—i.e., those in which patients were able to produce speech during only some tasks, or those that caused patients to have aphasia, dysnomias, paraphasic errors, or comprehension errors, were designated as LE nodes.

For intraoperative cases, after DES language mapping was completed, surface (subdural) electrode arrays were placed for data acquisition. The inventors visually co-registered the nearest surface electrodes with the previously identified positive mapping locations. For epilepsy patients, both electrodes in each bipolar stimulation pair were considered to be positive when a language deficit was elicited during stimulation.

All subjects performed a word-reading task in which they read single words from a screen while continuous ECoG was acquired. Although the timing and word set differed between subjects and across the two institutions, in general, each trial began with the display of a word on-screen for approximately two seconds. In some subjects, the word was read upon presentation. In others, a variable-length delay (blank screen) preceded a go cue prompting vocalization. Approximately five to seven minutes of this task from each participant was used for analysis, typically involving around 40-60 words.

During the testing, data was acquired at sampling rates ranging from 500 to 2 kHz. Notch filters were applied at 60, 90, and 120 Hertz (Hz) in a zero-phase manner and re-referenced data to the common mean. The Hγ power was extracted by applying eight finite impulse response band-pass filters with approximate pass-band widths of 10 Hz and linearly spaced band centers from 70-150 Hz, followed by a Hilbert transform to extract the analytic amplitude. After squaring the amplitude, the sub-band powers were averaged to obtain the Hγ power. In alternative embodiments, other methods of extracting Hγ power such as short-term Fourier transforms or wavelets, or powers in other frequency bands, may be used. At every step (i.e., after initial filtering, re-referencing, and after obtaining the Hγ power) of preprocessing, the data were visualized and electrodes whose signals were contaminated with frequent interictal spiking activity were excluded, as well as those with substantial noise or artifacts. After excluding noisy electrodes, the preprocessing pipeline was reiterated from the beginning without the excluded electrodes (thus preventing contamination in re-referencing) until no further noisy electrodes remained. For the remaining electrodes, the inventors identified and removed any transient epochs of significant artifact (defined as simultaneous high-amplitude aberrant activity across many electrodes) and concatenated the remaining data with a flat-top (trapezoidal) taper with linear transition zones of 100 milliseconds (ms).

Prior to network generation, the Hγ power was standardized using Z-scores. A global signal regression was also performed, thus ensuring that the residual data used to construct our networks was orthogonal to the global average. The resultant signal was smoothed by applying a 150 ms moving mean. This length was selected by visual inspection as one that reduced rapid fluctuations in the Hγ power signal while maintaining its underlying characteristics. In alternative embodiments, a different moving mean may be used.

A single network (i.e., graph) was constructed for each subject representing the functional connectivity during the entire task. In these networks, each node represented the activity in the cortex underlying an electrode. The inventors connected these nodes with edges based on the zero-lag, Fisher-transformed Pearson correlation of their Hγ power. The generated networks therefore contained edges ranging from [−1 1] in a weighted manner. Networks are described by their connectivity matrices, denoted here by the variable A where Aᵢⱼ represents the weight of connection between electrodes i and j. Thresholding of edges was avoided whenever possible. Instead, the edges characterized left as weighted positive and negative connections between nodes, as this reduced the potential parameterization bias in subsequent analyses. In alternative embodiments, unweighted graphs, or other measures of connectivity such as coherence, mutual information, or non-zero-lag correlations, may be used.

Modularity maximization was used to reveal communities within the generated networks. Communities are groups of nodes that are more strongly connected to one another than expected by chance. This was quantified through the modularity objective function Q, which quantifies the quality of a particular partition of nodes into communities: Q=Σᵢⱼ[Aᵢⱼ−Pᵢⱼ]δ(zᵢ,zⱼ). Here, Aᵢⱼ and Pᵢⱼ are the observed and expected weights of connections between nodes i and j, zᵢ is the community assignment of node i, and δ is the Kronecker delta function. The variable Q is therefore a summation of the difference in observed vs. expected connection strengths of all pairs of nodes assigned to the same community, across the entire network, and was consequently maximized when the network was partitioned into communities that were each connected internally in a maximally stronger fashion than would be expected.

The Louvain algorithm was used in order to reveal community partitions and assess modularity. The Louvain algorithm is non-deterministic, so a consensus clustering step was performed by repeating the community detection process 100 times (thus generating an allegiance matrix representing 100 potential community partitions), upon which the Louvain algorithm was again used in order to discover the most robust partition of communities across iterations.

To define the expected set of connection weights, the Potts (uniform) null model was used, in which all P_{i≠j} were set to the mean non-self-directed edge strength of the corresponding network. Intuitively, this means the discovered communities were more strongly internally connected than in a comparison network where all nodes were interconnected with a strength equal to the average of the network under investigation. In the testing, this yielded a relatively stable and useful number of communities in each network. The stability of the findings was tested by exploring a range of values for P_{i≠j}, which did not substantially change results, as discussed in more detail below.

The inventors also examined the community membership patterns of LE and SA nodes. In particular, an examination was conducted regarding whether critical nodes (LE and SA nodes taken together), LE nodes, or SA nodes co-localized in the same communities with other nodes of their own phenotype (i.e., critical with critical, LE with LE, and SA with SA). To accomplish this, the co-assignment % was calculated. For each subject, co-assignment % can be defined the percentage of all pairs of each node phenotype that were assigned to the same community. To test whether the co-assignment % was different than chance, the community labels were randomly permuted 1000 times, a process that preserves the relative size of each community and is therefore more rigorous than random community assignment. The true co-assignment % for each group was compared to that of the surrogate distribution. The inventors also calculated the cross-assignment %, which is the fraction of node pairs consisting of one LE and one SA node that were assigned to the same community, and compared the cross-assignment % to chance in the same manner.

It was hypothesized that a node that has connections across multiple different communities plays a different role in the network (and may be more critical to network function) than one that has strong connections only within its own community. To quantify this, the participation coefficient (pc) of each node was calculated, defined as

$${p}_{i} = 1 - {\sum }_{s = 1}^{C} {( \frac{{k}_{is}}{{k}_{i}} )}^{2} ,$$

where s∈{1, . . . , C} represents community assignment, kᵢ is the node's total degree (binary network) or strength (weighted network) and kᵢₛ is the node's total degree or weight of connections to community s. The value of pc is bounded between 0 (i.e., when the node only makes connections to a single community), and 1 (i.e., when the node's connections are uniformly distributed across many different communities). For the calculation using weighted networks, only the contribution of positive-strength edges to the participation coefficient was considered.

The inventors also investigated several other well-described graph metrics: node strength (degree centrality), eigenvector centrality, participation coefficient, clustering coefficient, and local efficiency, using code derived from the Brain Connectivity Toolbox. Node strength quantifies the sum of all weighted connections a node makes in the network. Eigenvector centrality simultaneously considers a node's number of connections as well as the relative importance of those connections. The clustering coefficient of a node describes the likelihood that any two of its connections (i.e., its neighbors) are also connected to each other, and local efficiency is a related measure that quantifies the shortest path lengths in the local neighborhood of a node. Local efficiency was calculated based on positive edges only by converting connection strengths to path-lengths via their inverse (Lᵢⱼ=Aᵢⱼ⁻¹); the remainder of calculations were based on the weighted adjacency matrix. To standardize comparisons between subjects with different numbers of nodes and connection strength characteristics, all network metrics were Z-scored for each subject.

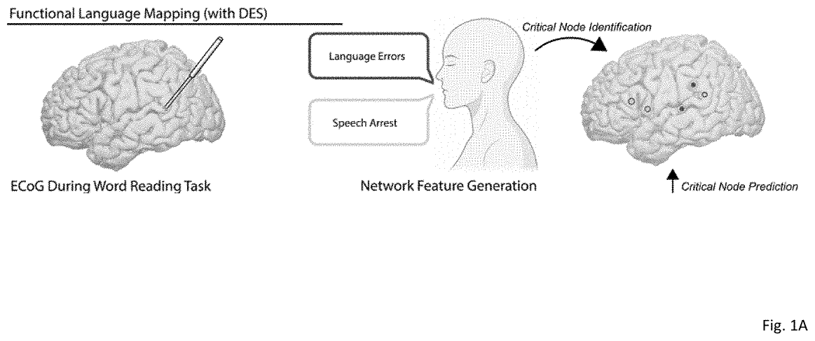

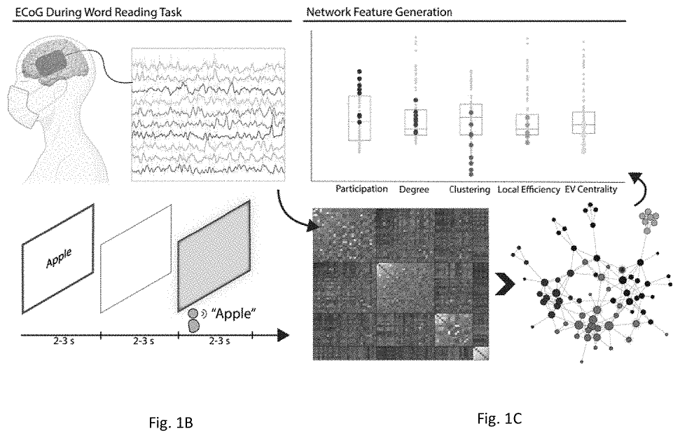

FIG. 1 depicts an overview of the experimental system used to identify networks. FIG. 1A depicts how DES is used either intraoperatively (depicted) or in the epilepsy monitoring unit to identify sites critical to language and speech in accordance with an illustrative embodiment. These were subdivided into regions causing speech arrest (SA) vs. other types of language errors (LE). FIG. 1B depicts ECoG recorded continuously during a simple word-reading task performed by each subject in accordance with an illustrative embodiment. FIG. 1C depicts how a single, static network for each patient was generated using the correlations between high gamma activity on each electrode in accordance with an illustrative embodiment. FIG. 1C also depicts how connectivity metrics were computed from the correlations. Community partitions were discovered (different shadings in lower right panel) using modularity maximization. Several well-known network metrics were calculated. These metrics were used to train machine learning classifiers to predict which nodes would be critical to language and speech, as discussed herein.

The inventors also investigated whether the network signatures alone could be used to predict whether nodes would be found to be critical by DES. As there was substantial heterogeneity in the number of grids, electrodes, coverage, and clinical characteristics of the underlying patients, machine learning classifiers were trained and evaluated within each subject using 10-fold cross-validation (assigning nodes randomly to each fold). This process roughly estimates the potential accuracy of these models in predicting critical nodes when trained and tested on data closely resembling that of each individual patient. Support vector machine (SVM) and k-nearest neighbor (KNN) classifiers were used due to their power, wide recognition, and straightforward implementation as representative machine learning models. Subjects with less than 4 nodes of the positive class were excluded due to class underrepresentation in training folds.

Within each of the 10 training and test fold pairs for each subject, model parameters (i.e., kernel type and size, cost-weighting of false negatives, etc.) were optimized by using Bayesian optimization to minimize an objective function of 1-AUC (area under curve of the receiver operating characteristic) on the training fold alone. This was done using 5-fold cross-validation.

The estimated optimal parameters thereby maximized the AUC across each of the 5 sub-test sets, which were completely independent from the test set used to estimate final classification accuracy. Using these parameters, classifiers were trained on the training set, and applied to the test set. Positive class prediction scores were used to generate receiver operating characteristic (ROC) curves. Optimal thresholds for each subject's ROC curve were defined as those maximizing balanced accuracy.

Using these same methods, the inventors further investigated whether language network signatures generalized across patients, by testing whether a classifier trained on a set of patients could be used to predict critical nodes in an entirely separate patient. This simulated the clinical scenario of using these network signatures to predict critical nodes in a new patient, which potentially could reduce the amount of stimulation involved for functional mapping, or possibly even augment it. Classifiers were trained in a leave-one-patient-out manner. For each patient, positive class prediction scores were again used to generate ROC curves, and optimal thresholds were defined to be those that maximized balanced accuracy.

The above process was repeated twenty times and the results averged to generate a point estimate of balanced accuracy, sensitivity, specificity, and average ROC for each subject. In order to compare the prediction accuracies to chance, a reference distribution was generated from twenty binomial predictions using the true positive class prior probability for each subject.

As noted, the above-discussed study included sixteen patients, including eleven patients with epilepsy and five patients who underwent awake craniotomy for tumor resection. Patients had a mean (±SD) of 77.5 (±34.6) intracranial subdural electrodes after exclusion of noisy electrodes. Fifteen (93.8%) patients had frontal electrodes, thirteen (81.3%) had temporal electrodes, eleven (68.8%) had parietal electrodes, and two (12.5%) had occipital electrodes. Patients had a mean of 9.9 (±4.1) electrodes that were labeled critical for speech or language using DES, of which 6.2 (±4.1) were LE nodes and 3.7 (±3.0) were SA nodes. There were, in total, 3176 electrodes, 115 critical nodes, 93 LE and 55 SA nodes.

With respect to community structure, a median 4 consensus communities (range 2-6) were discovered in each network. Communities contained a median 18.5 nodes [IQR 11-27]. Interestingly, SA and LE nodes were not randomly distributed across different communities. Rather, each critical node phenotype concentrated within its own community. It was found that 78.7% of SA and 62.2% of LE node pairs shared a community, compared to 61.0% and 48.9% in the permuted data, respectively (p<0.001 for both, permutation-based test). In contrast, the cross-assignment % between SA and LE nodes, nor the co-assignment % between critical nodes when both SA and LE nodes were pooled together, was different from chance.

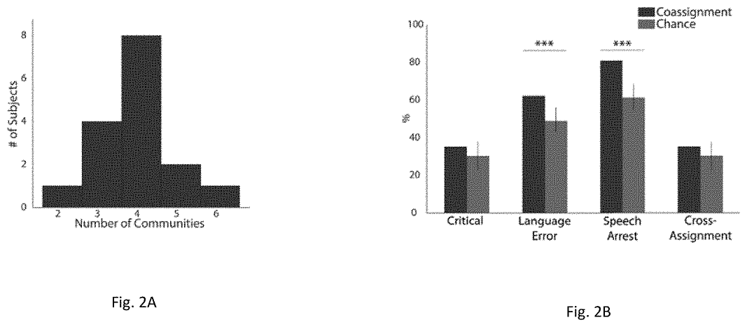

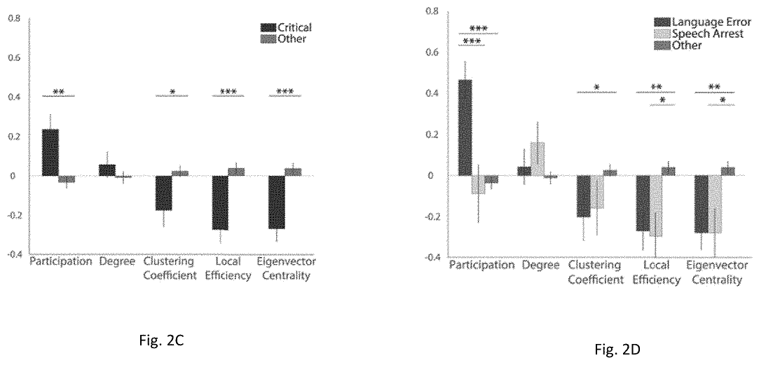

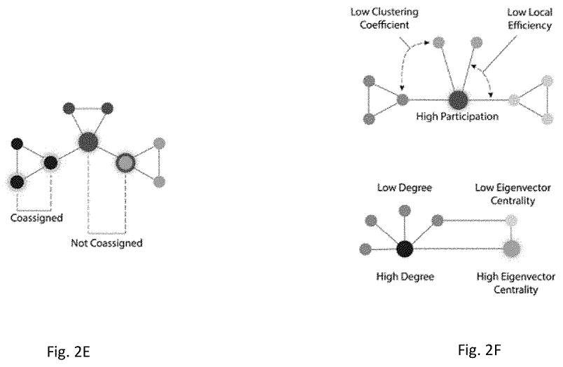

FIG. 2 depicts various communities and network metrics. Specifically, FIG. 2A is a histogram of the number of communities per patient in accordance with an illustrative embodiment. FIG. 2B depicts coassignment percentages and chance coassignment in accordance with an illustrative embodiment. It was found that language error nodes and speech arrest nodes are significantly more likely to coassign in the same communities than chance, whereas they were not more likely to be found in the same community as each other, or when pooled together as critical nodes. FIG. 2C shows network metrics for critical (LE+SA) nodes vs. other nodes in accordance with an illustrative embodiment. FIG. 2D depicts network metrics for LE nodes and SA nodes vs. other nodes in accordance with an illustrative embodiment. It is noted that the increase in participation coefficient in panel C is driven by a marked increase in participation for language error nodes. FIG. 2E is a first diagram explaining coassignment and other network metrics in accordance with an illustrative embodiment. FIG. 2F is a second diagram explaining coassignment and other network metrics in accordance with an illustrative embodiment.

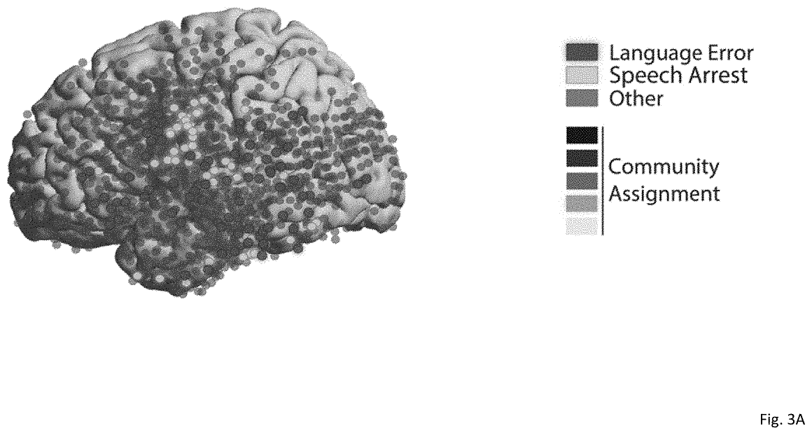

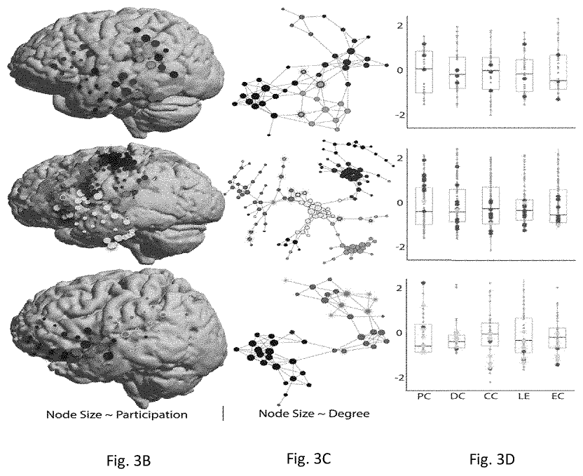

FIG. 3 is a depiction of patients' brain networks and anatomic electrode distribution. FIG. 3A depicts all electrodes from all patients on a single template brain in accordance with an illustrative embodiment. Speech arrest nodes are located in ventral prefrontal regions, but also in ventral temporal regions. Language error nodes are widely distributed in perisylvian regions. FIG. 3B depicts three example patient brain networks in accordance with an illustrative embodiment. Node shading (filled) represents community assignment, and node size is related to participation coefficient. Outline indicates critical nodes (blue (darker shade)— LE node, yellow (lighter shade)— SA node). FIG. 3C depicts network communities (in different shades of gray) from the three example patients in accordance with an illustrative embodiment. Electrode position is spring-weighted (stronger connections draw electrodes closer together). FIG. 3D depicts network metrics for the three example patients in accordance with an illustrative embodiment.

Critical nodes identified by DES exhibited significantly higher participation coefficients than did other nodes (FIG. 3A). Interestingly, it was found that the high pc was driven by a markedly high pc in LE, rather than SA nodes (FIG. 3B). To further investigate candidacy of these nodes as local hubs, their clustering coefficient and local efficiency were calculated (FIG. 3A). Critical nodes (both LE and SA nodes) were significantly lower in both measures than other nodes. Degree centrality was not found to be different between critical or LE nodes and other nodes, while eigenvector centrality was significantly lower in both SA and LE nodes than other nodes. Thus, critical nodes, and particularly language error nodes, were connectors between brain network modules, rather than local or global hubs.

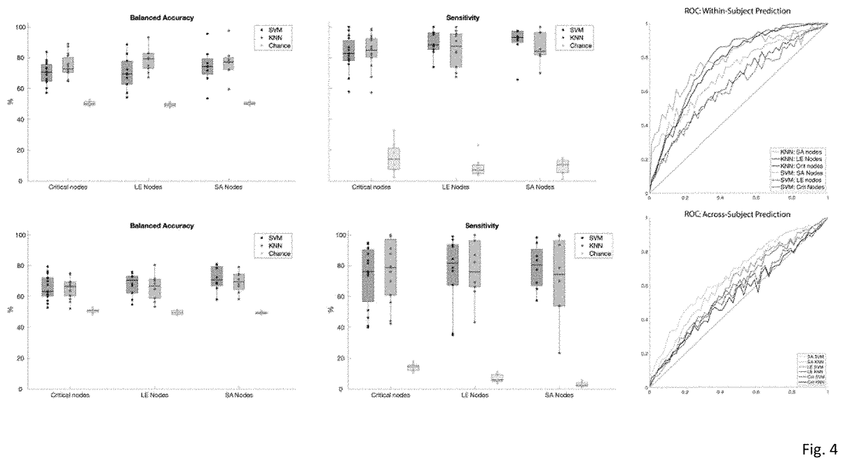

An assessment was also conducted regarding the ability of these network signatures to predict which nodes were critical to speech and language within each subject using 10-fold cross-validation. Average balanced accuracy and sensitivity were substantially and significantly better than chance. For critical nodes, median balanced accuracy was 70.6% and 72.8%, and median sensitivity was 82.8% and 85.0% for SVM and KNN, respectively, compared to 50.8% and 14.2% for chance (p<0.001 for all via rank sum test). When predicting LE nodes and SA nodes separately, balanced accuracy and sensitivity improved in nearly all models, with the best yielding 79.3% balanced accuracy and 88.3% sensitivity for LE nodes and 77.2% balanced accuracy and 93.3% sensitivity for SA nodes. FIG. 4 depicts balanced accuracy, sensitivity, and ROC curves for within-subject (top row) and across-subject (bottom row) classification in accordance with an illustrative embodiment. For each subject, an average receiver operating characteristic (ROC) curve was generated, for which the AUC was calculated.

Additional investigation was also conducted regarding whether language network signatures could be used to train a model that could predict critical nodes in an entirely new patient, using leave-one-patient-out cross-validation. For critical nodes, classification median balanced accuracy was 63.4% and 66.5% and median sensitivity was 76.3% and 78.8%, compared to 50.5% balanced accuracy and 15.0% sensitivity by chance (p<0.001, rank sum test). Again, when classifying LE and SA nodes separately, accuracy and sensitivity improved for most models, with the best yielding 70.5% balanced accuracy and 81.6% sensitivity for LE nodes and 70.8% balanced accuracy and 80.5% sensitivity for SA nodes.

For both within-subject and across-subject prediction, average ROC curves were generated and the average area under curve was calculated. The data shows the average (median) balanced accuracy and sensitivity for classifiers, tested via 10-fold cross validation. The highest performing model for each classification task (critical node, language error node, and speech arrest node) was determined. P values calculated via rank sum (non-parametric) test for twenty prediction attempts vs. twenty chance prediction attempts using the true positive class prior probability. Although accuracy and sensitivity tended to improve when classifying language error nodes and speech arrest nodes when compared to critical nodes, the differences were not statistically significant.

As discussed above, direct electrocortical stimulation is commonly used to identify cortical areas critical to speech and language function, yet it is not clear what makes those areas so important to the language network. As such, the inventors examined the network properties of these nodes using graph theory applied to ECoG recordings during a word-reading task. As discussed, it was found that nodes critical to speech and language differed from other nodes in multiple network metrics including their participation coefficient, clustering coefficient, local efficiency, and eigenvector centrality. In particular, two different network signatures were identified between nodes that, when stimulated by DES, cause either speech arrest or language errors. While both types similarly exhibited lower local clustering and eigenvector centrality, i.e., they were not provincial or global hubs—language error nodes in particular demonstrated higher participation coefficients, suggesting that they acted as connector nodes between communities in language networks. This finding offers insight into the network role of critical cortical areas that, when stimulated (and by extension, when lesioned) cause dysphasias, dysnomias, comprehension errors, or other fluent language deficits. In contrast, areas where DES resulted in speech arrest did not resemble connector nodes in the functional speech network. The interaction of these nodes with other network communities, such as those underlying speech motor articulation or gestures, can also be explored.

Human language is a complex phenomenon in which multiple disparate executive, memory, emotive, and sensorimotor systems integrate in a rapid multistage process. Leading hypotheses on the cognitive processes underlying speech and language have evolved from an emphasis on concentrated activity in specific brain regions to the perspective that language depends upon widespread activation of multiple cortical networks engaged in the parallel processing of language subtasks. However, significant gaps remain in the understanding of these networks and how they interact. The findings described herein identify a critical role for connector nodes in these networks. A small set of network features, generated from a single, static network estimated over the entirety of a simple word-reading task, were used to predict which nodes in the network are critical to speech and language function. This implies that the pattern of connections within the language network strongly informs the criticality of speech and language nodes. Further investigation into the networks underlying speech and language processing, in combination with additional spectral, anatomical, and task-based information can be used to increase prediction accuracies to levels that are clinically applicable.

It has been established that intermodular connections make nodes critical to language function. Brain networks have small-world topologies, in which more densely-connected local communities are interconnected by sparser long-range edges between communities. This architecture supports efficient information transfer while conserving the total number of connections. Thus, connector nodes are critical to this organization. In the above-discussed study, it was found that language-critical nodes had significantly higher pc than speech-critical nodes or other nodes. Concurrently, both LE and SA nodes had lower clustering coefficients and local efficiencies than other nodes, indicating that they were not local hubs (i.e., critical nodes' neighbors were themselves less strongly interconnected, and had longer relative path lengths between them than non-critical nodes' neighbors). Eigenvector centrality was lower for all critical nodes than noncritical nodes, while degree centrality was not different, suggesting that critical nodes did not simply strongly connect to the most strongly connected nodes in the network, and they were not themselves global or local hubs (i.e., were not simply highly connected overall). Rather critical nodes, and in particular LE nodes, were distinguished by the pattern of their connections and position within the network.

Taken together, these metrics form a network signature that strongly implicates language-critical nodes as connectors in the language network. This, in turn, implies that for higher-order cognitive functions such as language, which depend on coordinated actions of multiple subnetworks (communities), the connectors between these nodes are critical to the overall cognitive function. In contrast, for lower-order functions, such as speech articulation (identified via speech arrests), or possibly for motor function, connectors between communities are less critical. In the study, motor nodes were relatively underrepresented in electrode placement, and it may be that the test networks under-sampled the regions connected to by SA nodes.

As discussed herein, ECoG-based functional mapping using network signatures was performed. DES-based functional mapping is well-established to improve neurologic outcomes and safe extent of resection of tissue near eloquent areas. By improving the extent of resection, patients with brain tumors experience increased survival and those with epilepsy experience improved seizure control and quality of life. However, DES is time-consuming, can be tiring, and can increase risk to patients. DES parameters vary significantly between individuals as well as stimulation locations within one individual, and stimulation-evoked after-discharges are common and sometimes cause seizures. Furthermore, DES-based functional mapping requires an able and willing participant, and can be challenging in particular populations. Finally, DES mapping can both over- and under-estimate sites that are ultimately critical to brain function (i.e., its findings do not always match post-operative outcomes), suggesting it could be improved upon.

To address these limitations, mapping based on ECoG recording could be used to reduce the amount of stimulation required for mapping (and thereby reduce risk), shorten mapping times, and alleviate other factors such as the inability of some patients to participate. Prior studies of ECoG functional mapping have attempted to predict DES-defined language-positive nodes using Hγ modulation, network features combined with power, and deep-learning approaches using with multiple time- and spectral-domain features.

Using the framework described herein, the inventors predicted critical nodes with high balanced accuracy and sensitivity using only a limited set of network features with straightforward machine-learning classifiers. The results compare favorably with other studies using ECoG to predict critical nodes. This is true of both those using just the Hγ modulation in each electrode as a marker for spectral functional mapping and another study that used a combination of trial-averaged and stimulus or articulation-aligned power and network features. However, the findings discussed herein are remarkable in that the inventors used a small set of hub-related features derived from a single, static network generated to represent the functional connectivity over the entirety of a short task period, in a larger and more diverse set of patients, without further trial-averaging or sub-selecting time windows or electrode pairs based on task or stimulation parameters.

Unlike most prior analyses, the inventors also took the extra step of classifying across participants. A balanced classification accuracy as high as 70.8% with a sensitivity of over 80% was achieved. This approximates the accuracy one would achieve applying the trained model on a new, previously unseen patient, which is how ECoG mapping would likely be used in clinical practice. Ultimately, the combination of powerful network features (such as those described herein) combined with sophisticated deep learning approaches can yield clinically useful prediction accuracies.

Thus, the findings described herein provide a new insight into the different roles of language error and speech arrest nodes in language networks, and in particular highlight the eloquent role of connector nodes within these networks. The findings were robust across a reasonably large group of patients with diverse cortical sampling, and a range for the community resolution parameter was tested to decrease the chance this finding was due to coincidence. However, functional networks were constructed using high-gamma correlations alone, and the functional networks only sampled the portions of language networks colocalized under the electrode grids and strips placed for clinical purposes in these patients.

The approach towards network generation was to create a single, static network representing the entirety of the task. While this approach is attractive due to its simplicity and potential real-world ease of implementation for passive functional mapping, language is a dynamic process integrating activity from multiple areas and subnetworks. The vast majority of the study of these networks has been through fMRI, with a minimum time resolution spanning at least several seconds. It is unknown whether language subnetworks revealed by fMRI remain largely persistent during this time, or whether they reflect the summation of the rapid reconfiguration of networks serving multiple language subprocesses.

In the static networks, two different network signatures were discovered for nodes causing language errors and speech arrest. It is possible that language is subserved by dynamic networks, and that DES may actually identify nodes that play different, important roles during language processing. These might be further elucidated experimentally with both static and dynamic network analyses during more diverse and targeted linguistic tasks (e.g., semantic recall, comprehension, spontaneous speech, etc.). These dynamic network changes during language production are an area of active investigation.

Based on the above-discussed study, the inventors have developed methods and systems for improving functional brain mapping. The proposed methods and systems use information from multiple modalities, including brain imaging and electrical recordings, to map brain function. Based on this information from multiple modalities, the critical areas of the brain are predicted by using the network properties of the brain to establish how they are connected to other areas of the brain. This technology can help to improve functional outcomes for people with brain tumors by improving the accuracy of cortical mapping and reducing the intraoperative time. The proposed system uses mathematical techniques applied to brain recordings and imaging to estimate functional mapping without using stimulation.

In an illustrative embodiment, the brain can be thought of as a network that includes two fundamental components: nodes (e.g., individual areas of gray matter, which can be analyzed with electrocorticography (ECoG) electrodes) and edges that connect node pairs (white matter). The connectivity among brain areas can be described using either structural networks or functional networks. In structural networks, the edges represent physical connections (e.g., white matter tracts) between nodes. In contrast, the edges in functional networks represent correlations or coherence between the activity recorded from separate brain areas. This connectivity can be static (over all recording time) or dynamic (different connectivity matrices at different points in time). In one embodiment, dynamic connectivity can be represented as a series of network layers, each of which represents connectivity for a given time window. The proposed system utilizes these frameworks to perform functional brain mapping.

In one embodiment, the proposed system obtains/receives a combination of recordings from intracranial electrodes (electrocorticography (ECoG) and/or stereo-electroencephalography (sEEG)), magnetic resonance imaging (MRI) (including functional MRI (fMRI) during task and/or resting state, as well as structural and diffusion-weighted imaging and tractography) from individual patients. The ECoG and fMRI measurements can be recorded while the patient is performing tasks involving speech, language, or movement of (or somatosensory stimulation of) the limbs or face, and also while the patient is in a resting state (i.e., not performing a task). In some embodiments, the task performed by the patient can also be a cognitive task, such as attention or memory tasks.

The data obtained/received by the system is then processed to determine the edge network connectivity (either structural and/or functional) between cortical nodes. For MRI, the nodes are voxel parcels (groups of highly correlated voxels smaller than standard regions of interest) on the millimeter (mm) scale. In the case of electrical recordings, the nodes are the extracted frequency band powers in multiple frequency bands, including delta, theta, alpha, beta, low gamma, and high gamma bands, or the time domain signals. In the case of functional connectivity (for ECoG, sEEG, and fMRI, or corticocortical evoked potentials (CCEPs)), this can be computed as the pairwise correlation coefficients (or coherence or mutual information) between activity in all nodes.

Once the edge networks have been computed, the system computes connectivity metrics from graph theory, including but not limited to measures of centrality, degree, strength, and participation coefficient (community detection), for each of the edge networks. For a given network, communities are defined as groups of nodes that are more strongly connected to one another than expected by chance. Participation coefficient and flexibility measure the distribution of critical nodes across versus within communities for static and dynamic networks, respectively. In an illustrative embodiment, the system then uses these metrics as features in machine learning classifiers to classify each node as critical or not critical to the particular function being mapped.

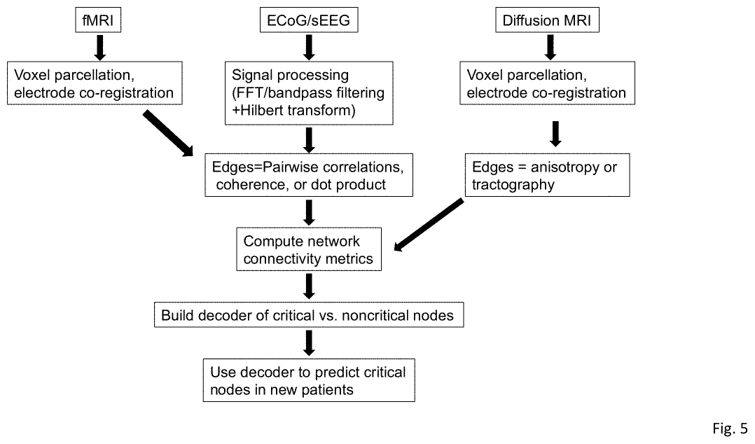

FIG. 5 is a flow diagram depicting operations performed by the system in accordance with an illustrative embodiment. As shown, the system performs various tests on the patient to obtain brain data. These tests include functional MRI (fMRI), the use of ECoG electrodes and/or stereo0electroencephalogram (sEEG) electrodes, and diffusion MRI (including but not limited to diffusion tensor imaging and diffusion spectral imaging). In alternative embodiments, fewer, additional, and/or different tests may be performed to obtain the brain data. Voxel parcellation is performed on the diffusion MRI and fMRI data to categorize the brain based on voxels. Electrode co-registration is also performed to map the fMRI and diffusion MRI coordinates to the locations of ECoG and/or sEEG electrodes. Signal processing is performed on the signals received from the ECoG/sEEG electrodes. The signal processing can include fast Fourier transform (FFT), bandpass filtering plus a Hilbert transform, or wavelet transform to produce frequency band powers such that the received signals are usable by the system.

The fMRI data and the ECoG/sEEG data are used by the system to identify network edges in the brain. The network edges are identified based on pairwise correlations, coherence, and/or dot product manipulations. The diffusion MRI data is also used by the system to identify network edges in the brain. The network edges are identified using tractography and/or anisotropy characteristics obtained from the diffusion MRI data. The network edge data based on the fMRI, diffusion MRI, and ECoG/sEEG data are all analyzed by the system to produce connectivity metrics. After analyzing the edge data with connectivity metrics, the system uses this information to build a decoder of critical versus non-critical brain nodes. The system is able to use this decoder to predict critical nodes in new patients.

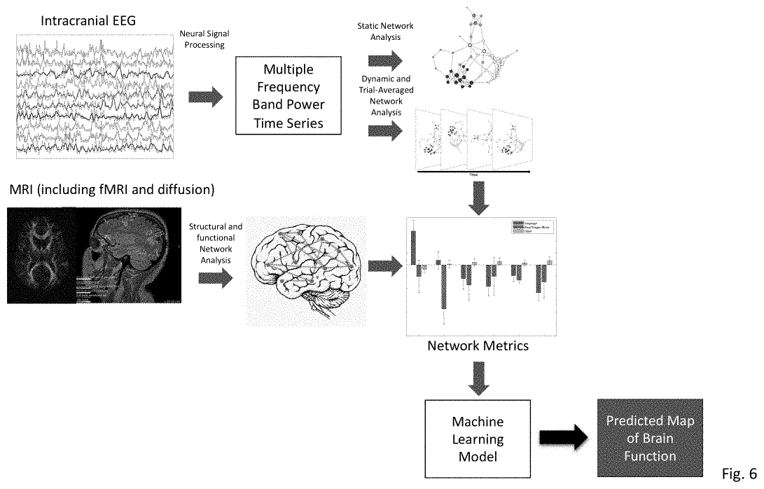

FIG. 6 is a flow diagram depicting operations performed by the system in accordance with an illustrative embodiment. In the depicted embodiment, the system receives data from an intracranial EEG (ECoG and/or sEEG) and from MRI (including both fMRI and diffusion MRI). In alternative embodiments, fewer, additional, and/or different measurements may be performed to source the data. Neural signal processing is performed on the intracranial EEG data, and the neural signal processing can include computing multiple frequency band power time series. The processed intracranial EEG data is also subjected to static network analysis, dynamic network analysis, and/or trial-averaged network analysis to generate network metrics based on the data.

The MRI data (including both the fMRI data and the diffusion MRI data) are subjected to structural and functional network analysis to generate additional network metrics. A machine learning model is used to combine the network metrics based on the intracranial EEG data and the network metrics based on the MRI data to generate a predicted map of brain function. The generated map can be specific to an individual patient, in some embodiments. In other embodiments, the generated map be used to predict brain function mapping for any patient.

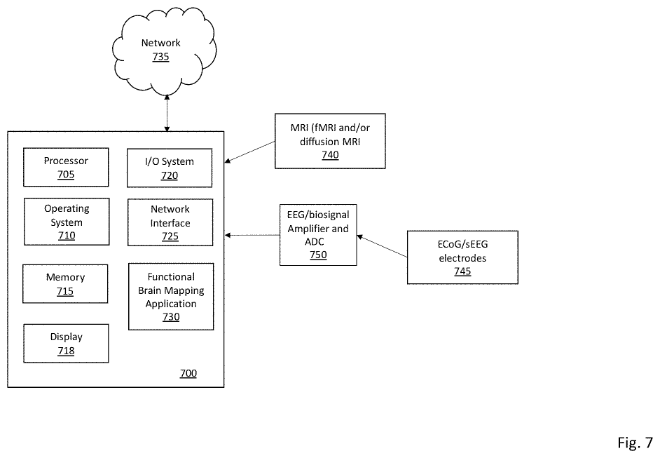

FIG. 7 depicts a computing system 700 for performing functional brain mapping in accordance with an illustrative embodiment. In the depicted embodiment, the computing system 700 in communication with a network 735, an MRI system 740 (including both fMRI and/or diffusion MRI), and ECoG/sEEG electrodes 745. In an illustrative embodiment, the computing system 700 receives measured data directly from the MRI system 740 and from the ECoG/sEEG electrodes 745 via an EEG/biosignal amplifier and analog-to-digital converter (ADC) 750. Specifically, data from the electrodes can go through the EEG/biosignal amplifier and ADC 750 for amplification and digitization prior to being received by the computing system 700. The computing system 700 can also receive measured data indirectly through the network 735.

The computing system 700 (i.e., functional brain mapping system) includes a processor 705, an operating system 710, a memory 715, a display 718, an input/output (I/O) system 720, a network interface 725, and a functional brain mapping application 730. In alternative embodiments, the computing system 700 may include fewer, additional, and/or different components. The components of the computing system 700 communicate with one another via one or more buses or any other interconnect system. The computing system 700 can be any type of computing system (e.g., smartphone, tablet, laptop, desktop, etc.), including a dedicated standalone computing system that is designed to perform the mapping.

The processor 705 can be in electrical communication with and used to control any of the system components described herein. For example, the processor can be used to execute the functional brain mapping application 730, process received user MRI data and electrode data, perform mapping, display results, etc. The processor 705 can be any type of computer processor known in the art, and can include a plurality of processors and/or a plurality of processing cores. The processor 705 can include a controller, a microcontroller, an audio processor, a graphics processing unit, a hardware accelerator, a digital signal processor, etc. Additionally, the processor 705 may be implemented as a complex instruction set computer processor, a reduced instruction set computer processor, an x86 instruction set computer processor, etc. The processor 705 is used to run the operating system 710, which can be any type of operating system.

The operating system 710 is stored in the memory 715, which is also used to store programs, received measurements/data, network and communications data, peripheral component data, the functional brain mapping application 730, and other operating instructions. The memory 715 can be one or more memory systems that include various types of computer memory such as flash memory, random access memory (RAM), dynamic (RAM), static (RAM), a universal serial bus (USB) drive, an optical disk drive, a tape drive, an internal storage device, a non-volatile storage device, a hard disk drive (HDD), a volatile storage device, etc. In some embodiments, at least a portion of the memory 715 can be in the cloud to provide cloud storage for the system. Similarly, in one embodiment, any of the computing components described herein (e.g., the processor 705, etc.) can be implemented in the cloud such that the system can be run and controlled through cloud computing.

The I/O system 720 is the framework which enables users and peripheral devices to interact with the computing system 700. The display 718 can include a touch screen in some embodiments, and the touch screen can be part of the I/O system 720 that allows a user to make selections, control sub-systems, view maps, etc. The display 718 can be any type of display, including a monitor, projector, virtual reality, or augmented reality display (e.g., the brain mapping can be displayed as an overlay on top of the actual brain), and can be used to present user interface screens and measured readings and/or the functional map to the physician. The I/O system 720 can also include one or more speakers, one or more microphones, a keyboard, a mouse, one or more buttons or other controls, etc. that allow the user to interact with and control the computing system 700. The I/O system 720 also includes circuitry and a bus structure to interface with peripheral computing devices such as power sources, universal service bus (USB) devices, data acquisition cards, peripheral component interconnect express (PCIe) devices, serial advanced technology attachment (SATA) devices, high definition multimedia interface (HDMI) devices, proprietary connection devices, etc.

The network interface 725 includes transceiver circuitry (e.g., a transmitter and a receiver) that allows the computing system 700 to transmit and receive data to/from other devices such as remote computing systems, servers, websites, etc. The network interface 725 enables communication through the network 735, which can be one or more communication networks. The network 735 can include a cable network, a fiber network, a cellular network, a wi-fi network, a landline telephone network, a microwave network, a satellite network, etc. The network interface 725 also includes circuitry to allow device-to-device communication such as Bluetooth® communication.

The functional brain mapping application 730 can include software and algorithms in the form of computer-readable instructions which, upon execution by the processor 705, performs any of the various operations described herein such as receiving measured data from external systems or elsewhere, performing voxel parcellation, performing electrode co-registration, signal processing including fast Fourier transform (FFT), bandpass filtering, and/or Hilbert transformation, identification of network edges in the brain based on pairwise correlations, coherence, and/or dot product manipulations, identification of network edges using tractography and/or anisotropy characteristics obtained from the diffusion MRI data, application of connectivity metrics to the network edges (these metrics can be static, dynamic, and/or based on trial-averaged network edges), performing structural and functional network analysis on the MRI data to generate additional network metrics, training and using a machine learning decoder model to combine the network metrics and generating a predicted map of critical vs. noncritical nodes, etc. The decoder can use, for example, a support vector machine, k-nearest neighbors, bagged trees, artificial neural network, or other classification algorithm. The functional brain mapping application 730 can utilize the processor 705 and/or the memory 715 and/or the display 718 as discussed above. In an alternative implementation, the functional brain mapping application 730 can be remote or independent from the computing system 700, but in communication therewith.

In summary, the inventors were able to classify critical language and speech nodes with remarkable accuracy using a small set of network-derived features. It was found that language error and speech arrest nodes have different network signatures, with the former acting as connector nodes in the language network. These network signatures alone are able to identify these nodes with a high degree of accuracy.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more”.

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

What is claimed is:

1. A system for performing functional brain mapping, the system comprising:
a memory configured to store first data from a magnetic resonance imaging (MRI) system and second data from one or more electrodes; and
a processor operatively coupled to the memory and configured to:
identify first edges in a brain network based on the first data from the MRI and second edges in the brain network based on the second data from the one or more electrodes;
determine, based on the first edges and the second edges, network connectivity metrics for the brain network; and
generate, based at least in part on the network connectivity metrics, a decoder that differentiates between critical nodes and non-critical nodes in the brain network.

2. The system of claim 1, wherein the MRI data includes diffusion MRI data.

3. The system of claim 1, wherein the one or more electrodes comprise stereo-electroencephalography electrodes.

4. The system of claim 1, wherein the one or more electrodes comprise electrocorticography electrodes.

5. The system of claim 1, wherein the processor is configured to generate a brain function map with the decoder, wherein the brain function map depicts the critical nodes and the non-critical nodes.

6. The system of claim 1, wherein the processor performs tractography on the first data to identify the first edges in the brain network.

7. The system of claim 1, wherein the processor performs voxel parcellation and electrode co-registration on the first data.

8. The system of claim 1, wherein the processor is also configured to determine first network connectivity metrics based on the first data and second connectivity network metrics based on the second data.

9. The system of claim 8, wherein the processor is configured to determine third network connectivity metrics based on third data from the MRI system and fourth connectivity network metrics based on fourth data from the electrodes, wherein the first data is diffusion MRI data, the second data is stereo-electroencephalography electrode data, the third data is functional MRI data, and the fourth data is electrocorticography electrode data.

10. The system of claim 1, wherein the network connectivity metrics are static, dynamic, or based on time-averaged data.

11. The system of claim 1, wherein the network connectivity metrics include one or more of local efficiency, participation coefficient, and clustering coefficient.

12. A method of performing functional brain mapping, the method comprising:
storing, in a memory, first data from a magnetic resonance imaging (MRI) system and second data from one or more electrodes; and
identifying, by a processor in communication with the memory, first edges in a brain network based on the first data from the MRI and second edges in the brain network based on the second data from the electrodes;
determining, by the processor and based on the first edges and the second edges, network connectivity metrics for the brain network; and
generating, by the processor and based at least in part on the network connectivity metrics, a decoder that differentiates between critical nodes and non-critical nodes in the brain network.

13. The method of claim 12, further comprising generating, by the processor, a brain function map with the decoder.

14. The method of claim 13, wherein the brain function map includes a plurality of nodes, and further comprising identifying, by the processor, one or more of the nodes as critical nodes.

15. The method of claim 14, further comprising identifying, by the processor, the critical nodes as language error (LE) nodes, speech arrest (SA) nodes, motor nodes, somatosensory nodes, nodes critical to memory, or nodes critical to higher cognitive functions.

16. The method of claim 14, further comprising calculating high-gamma correlations to identify the nodes as critical nodes.

17. The method of claim 16, further comprising recording, by the processor, local field potentials during a task performed by a patient, wherein the high-gamma correlations are calculated based at least in part on the local field potentials.

18. The method of claim 12, wherein the network connectivity metrics include one or more of local efficiency, participation coefficient, clustering coefficient, and flexibility.

19. The method of claim 12, wherein identifying the first edge in the brain network comprises performing, by the processor, tractography on the first data to identify the first edges.

20. The method of claim 12, further comprising performing, by the processor, voxel parcellation and electrode co-registration on the first data.
